SEQUENCE LISTING (1 sequence)

SEQ ID NO: 1 - protein, 37 aa, Homo sapiens [sequence omitted]

(19) United States
(12) Patent Application Publication
(10) Pub. No.: US 2010/0222251 A1
(43) Pub. Date: Sep. 2, 2010

(54) MIXTURE COMPRISING AN AMYLIN PEPTIDE AND A PROTRACTED INSULIN

(75) Inventor: Morten Schlein, Vaerlose (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/676,770

(22) PCT Filed: Sep. 11, 2008
(86) PCT No.: PCT/EP2008/062034

§ 371 Date: Apr. 22, 2010

Related U.S. Application Data
(60) Provisional application No. 60/972,262, filed on Sep. 14, 2007.

(30) Foreign Application Priority Data
Sep. 11, 2007 (EP) 07116065.9

(51) Int. Cl. A61K 38/28; A61P 3/10; A61P 3/00

(52) U.S. Cl. 514/4

(57) ABSTRACT

The invention relates to a soluble pharmaceutical composition for parenteral administration, which comprises an amylin peptide and a protracted insulin peptide.

### FIELD OF THE INVENTION

The invention relates to a soluble pharmaceutical composition for parenteral administration, which comprises an amylin peptide and a protracted insulin peptide.

### BACKGROUND OF THE INVENTION

Diabetes mellitus is a metabolic disorder in which the ability to utilize glucose is partly or completely lost.

In the treatment of diabetes mellitus, many varieties of insulin formulations have been suggested and used. Some of the commercial available insulin formulations are characterized by a fast onset of action and other formulations have a relatively slow onset but show a more or less prolonged action.

Human insulin consists of two polypeptide chains, the so-called A and B chains which contain 21 and 30 amino acid residues, respectively. The A and B chains are interconnected by two cystine disulphide bridges. Within the last decade a number of human insulin analogues have been developed. They are designed for particular profiles of action, i.e. fast acting or prolonged action.

Another peptide of interest in the treatment of diabetes is amylin. Human amylin is a 37 amino acid long peptide which has physico-chemical properties that make its use as a drug troublesome. In particular, it has a tendency to fibrillate ex-vivo and become ineffective due to precipitation. There is currently on the marked a drug product called Symlin® containing an analogue of human amylin (pramlintide) where the three amino acids in positions 25, 28 and 29 each are substituted to proline. This improves substantially the fibrillating tendency. However, even pramlintide is difficult to keep in solution at neutral pH and it is therefore provided in an acidic solution i.e. Symlin®.

Symlin® is approved as an adjunct drug with insulin. Symlin® is currently administered as a separate injection at a separate injection site three times daily. If the patient also uses three insulin injections per day this adds to a total of six daily injections.

It would be useful to provide a pharmaceutical composition combining an amylin peptide, and a protracted insulin peptide in a stable solution in order to be able to better mimic the physiological profile of the peptides in a patient in response to glucose metabolism and limit the number of daily injections.

### SUMMARY OF THE INVENTION

The invention relates to a soluble pharmaceutical composition for parenteral administration, which comprises an amylin peptide, and a protracted insulin peptide.

The invention also relates to a soluble pharmaceutical composition for parenteral administration, which comprises an amylin peptide and a protracted insulin peptide, wherein the pH of said pharmaceutical composition or a reconstituted solution of said pharmaceutical composition is from about pH 3.5 to about pH 5.5

The invention also relates to a soluble pharmaceutical composition for parenteral administration, which comprises an amylin peptide and a protracted insulin peptide, wherein the protracted insulin peptide has a time-action of more than 8 hours.

Further described is a method for treatment of hyperglycemia comprising parenteral administration of an effective amount of the pharmaceutical composition according to the invention and a pharmaceutical composition comprising a protracted insulin peptide and an amylin peptide according to the invention for use as a medicament in the treatment of hyperglycemia, binge eating and/or bulimia.

### DESCRIPTION OF THE DRAWINGS

FIG. 1 shows the solubility of a formulation of GlyA21 ArgB31 ArgB32 human insulin versus pH. The formulation consisted of 0.6 mM GlyA21 ArgB31 ArgB32 human insulin, 0.46 mM Zn(Ac)₂, 30 mM phenol. GlyA21 ArgB31 ArgB32 human insulin was completely precipitated above pH 6.5

FIG. 2 shows the solubility of a mix formulation of 0.6 mM GlyA21 ArgB31 ArgB32 human insulin and 50 μM of the amylin analogue pramlintide versus pH. The concentration of GlyA21 ArgB31 ArgB32 human insulin in solution versus pH was plotted with black lines and squares using the left y-axis; the concentration of pramlintide in solution versus pH was plotted with light grey lines and diamonds using the right y-axis.

FIG. 3 shows the solubility of a mix formulation of 0.6 mM GlyA21 ArgB31 ArgB32 human insulin and 100 μM pramlintide versus pH. The concentration of GlyA21 ArgB31 ArgB32 human insulin in solution versus pH was plotted with black lines and squares using the left y-axis; the concentration of pramlintide in solution versus pH was plotted with light grey lines and diamonds using the right y-axis.

FIG. 4 illustrates the physical stability of a pramlintide formulation similar to the commercially available Symlin® formulation as assessed using a ThT fibrillation assay.

FIG. 5: The physical stability of a pramlintide formulation (5-1), a GlyA21 ArgB31 ArgB32 human insulin formulation (5-2) and a mix formulation (5-3) containing both pramlintide and GlyA21 ArgB31 ArgB32 human insulin as compared at pH 4 using the ThT fibrillation assay.

FIG. 6: The physical stability of two GlyA21 ArgB31 ArgB32 human insulin formulations without (6-1) and with pramlintide (6-2) as compared in a ThT fibrillation assay.

### DEFINITIONS

When used herein the term “protracted insulin” or “protracted insulin peptide” shall mean an insulin analogue or an insulin derivative which has a controlled/sustained/retarded time-action in standard models of diabetes e.g. pharmacokinetic disappearance and/or appearance in pigs.

With “insulin” as used herein is meant human insulin, porcine insulin or bovine insulin with disulfide bridges between CysA7 and CysB7 and between CysA20 and CysB19 and an internal disulfide bridge between CysA6 and CysA11.

By “insulin analogue” as used herein is meant a polypeptide which has a molecular structure which formally can be derived from the structure of a naturally occurring insulin, for example that of human insulin, by deleting and/or exchanging at least one amino acid residue occurring in the naturally occurring insulin and/or adding at least one amino acid residue. The added and/or exchanged amino acid residues can either be codable amino acid residues or other naturally occurring residues or purely synthetic amino acid residues. In aspects of the invention a maximum of 10 amino acids have been modified. In aspects of the invention a maximum of 8, 7, 6, 5, 4, 3, 2 or 1 amino acids have been modified.

By “insulin derivative” as used herein is meant a naturally occurring insulin or an insulin analogue which has been chemically modified, e.g. by introducing a side chain in one or more positions of the insulin backbone or by oxidizing or reducing groups of the amino acid residues in the insulin or by converting a free carboxylic group to an ester group or to an amide group. Other derivatives are obtained by acylating a free amino group or a hydroxy group.

With “desB30 insulin”, “desB30 human insulin” is meant a natural insulin or an analogue thereof lacking the B30 amino acid residue.

With “B1”, “A1” etc. is meant the amino acid residue at position 1 in the B-chain of insulin (counted from the N-terminal end) and the amino acid residue at position 1 in the A-chain of insulin (counted from the N-terminal end), respectively. The amino acid residue in a specific position may also be denoted as e.g. PheB1 which means that the amino acid residue at position B1 is a phenylalanine residue.

By “amylin peptide” as used herein is meant amylin, an amylin analogue, an amylin derivative or an amylin agonist.

“Amylin” as used herein refers to a human peptide hormone of 37 amino acids referred to as amylin, which is co-secreted with insulin from β-cells of the pancreas. Human amylin has the following amino acid sequence: Lys-Cys-Asn-Thr-Ala-Thr-Cys-Ala-Thr-Gln- Arg-Leu-Ala-Asn-Phe-Leu-Val-His-Ser-Ser-Asn-Asn-Phe-Gly-Ala-lle-Leu-Ser-Ser-Thr-Asn-Val-Gly-Ser-Asn-Thr-Tyr (SEQ ID NO:1). Thus, the structural formula is Lys-Cys-Asn-Thr- Ala-Thr-Cys-Ala-Thr-Gln-Arg-Leu-Ala- Asn-Phe-Leu-Val-His-Ser-Ser-Asn-Asn-Phe-Gly-Ala-Ile-Leu-Ser-Ser-Thr-Asn-Val-Gly-Ser-Asn-Thr-Tyr-NH₂(SEQ ID NO: 1) with a disulfidebridge between the two Cys residues and an amide group attached to the C-terminal amino acid via a peptide bond. The term also includes variants of amylin as present in, and in isolatable form, other mammalian species. With respect to a naturally occurring amylin compound, the term includes such a compound in an isolated, purified, or other form that is otherwise not found in nature.

An “agonist” of amylin refers to a compound that mimics one or more effects (or activity) of amylin in vitro or in vivo. The effects of amylin include the ability to directly or indirectly interact or bind with one or more receptors that are activated or deactivated by amylin, for example, the receptor binding assay and the soleus muscle assay described in Examples 2 and 3, respectively in WO 2004/037168. Preferably, the amylin agonist is not a calcitonin, which, as used herein, refers to the human peptide hormone calcitonin and species variations of it, such as that of rat, salmon 10 and eel (including aminosuberic eel calcitonin).

Preferred amylin agonists may also be compounds having at least 60, 65, 70, 75, 80, 85, 90, 95, or 99% amino acid sequence identity to SEQ ID NO: 1 and having amylin activity.

Exemplary amylin agonist analogs contemplated in the use of the invention include those described in U.S. Pat. No. 5,686,411, U.S. Pat. Nos. 6,114,304, and 6,410,511, (Amylin Pharmaceuticals Inc), which are herein incorporated by reference in their entirety.

An “analog” or “analogue” or “agonist analog” of amylin refers to a compound that is similar in structure (e.g., derived from the primary amino acid sequence of amylin by substituting one or more natural or unnatural amino acids or peptidomimetics) to amylin and mimics an effect of amylin in vitro or in vivo.

The nomenclature of various amylin analogs useful in the present invention can be used to indicate both the peptide that the sequence is based on and the modifications made to any basic peptide amylin sequence, such as human amylin. An amino acid followed by a number or an amino acid preceded by a superscript number indicates that the named amino acid replaces the amino acid normally present at the amino acid position of the number/superscript number in the basic amino acid sequence. For example “Arg18 Pro25 Pro 28-h-amylin” and “¹⁸Arg^{25,28}Pro-h-amylin” refers to a peptide based on the sequence of “h-amylin” or “human-amylin” having the following substitutions: Arg replacing His at residue 18, Pro replacing Ala at residue 25 and Pro replacing Ser at residue 28. The terms “des-Lys1-h-amylin” and “des-¹Lys-h-amylin” refers to a peptide based on the sequence of human amylin, with the first, or N-terminal, amino acid deleted. Amylin analogs useful according to the invention may also include fragments of amylin such as those described in EP 289287, the contents of which are herein incorporated by reference.

Amylin analogs also include amylin having insertions, deletions, and/or substitutions in at least one or more amino acid positions of SEQ ID NO: 1. The number of amino acid insertions, deletions, or substitutions may be at least 1, 2, 3, 4, 5, 6, 10. Insertions or substitutions may be with other natural or unnatural amino acids, synthetic amino acids, peptidomimetics, or other chemical compounds.

Exemplary compounds include, but are not limited to des-Lys1-h-amylin, Pro28-h-amylin, Pro25 Pro28 Pro29-h-amylin, Arg18 Pro25 Pro28-h-amylin, Pro25 Val26 Pro 28 Pro29-h-amylin and Arg18 Pro25 Pro 28-des-Lys1-h-amylin, which all show amylin activity in vivo in treated test animals, (e.g. provoking marked hyperlactemia followed by hyperglycemia). In addition to having activities characteristic of amylin, certain of the preferred compounds of the invention have also been found to possess more desirable solubility and stability characteristics when compared to human amylin.

A “derivative” of amylin refers to an amylin which is chemically modified, e.g. by introducing a side chain in one or more positions of the amylin backbone or by oxidizing or reducing groups of the amino acid residues in the amylin or by converting a free carboxylic group to an ester group or to an amide group. Other derivatives are obtained by acylating a free amino group or a hydroxy group. Examples of amylin derivatives are described in the international patent application WO 2007/104789 (Novo Nordisk A/S) and pending European patent application no. 07116067.5. Further examples of amylin derivatives are N- methylated amylin, such as the amylin peptide described in Yan et al, PNAS, vol. 103, no. 7, p. 2046-2051, 2006, where the amylin is N-methylated in positions 24 and 26.

### DESCRIPTION OF THE INVENTION

In one aspect, the invention relates to a soluble pharmaceutical composition for parenteral administration, which comprises an amylin peptide, and a protracted insulin peptide.

In one aspect of the invention, the amylin peptide is amylin, an amylin analogue, an amylin derivative or an amylin agonist.

In one aspect of the invention, said amylin peptide is human amylin. In one aspect of the invention, said amylin peptide is Pro25 Pro28 Pro29-h-amylin (pramlintide). In a further aspect of the invention, said amylin peptide is human amylin methylated in position 24 and 26.

In one aspect of the invention the amylin peptide is selected from the group of amylin derivatives described in patent application WO 2007/104789 (Novo Nordisk A/S). In one aspect of the invention the amylin peptide is selected from the group of amylin derivatives described in European patent application No. 07116067.5 (Novo Nordisk A/S).

It is understood that biological active amylin agonists may have an amide group attached to the acid group of the C terminal residue via a peptide bond. In one aspect an amylin of the invention is human amylin or an amylin analogue which has have an amide group attached to the acid group of the C terminal residue via a peptide bond.

In another aspect of the invention, the concentration of said amylin peptide in said pharmaceutical composition is in the range from about 0.2 mg/mL to about 1.2 mg/mL. In a further aspect the concentration of said amylin is from about 0.2 mg/mL to about 0.8 mg/mL. In a yet further aspect the concentration of said amylin peptide is from about 0.3 mg/mL to about 0.7 mg/mL. In a further aspect the concentration of said amylin peptide is from about 0.2 mg/mL to about 0.7 mg/mL. In a yet further aspect the concentration of said amylin peptide is from about 0.2 mg/mL to about 0.4 mg/mL.

The amylin peptide of the present invention may be capable of binding to or otherwise directly or indirectly interacting with an amylin receptor, or other receptor or receptors with which amylin itself may interact to elicit a biological response, e.g., reducing food intake. Compounds of the invention may bind an amylin receptor with an affinity of greater than 20 nM, 10 nM, 5 nM, and more preferably with an affinity of greater than 0.10 nM which may be determined by an amylin receptor assay such as e.g. described below:

For the receptor binding assay, membranes from the Amylin 3(a)/CRE-luc cells may be used. The tracer is Tyr-pramlintide iodinated with ¹²⁵I in the N-terminal tyrosine. SPA-WGA beads (GE Healthcare RPNQ0001) are incubated in a 96 well Optiplate in a buffer containing 50 mM Hepes, 5 mM MgCl₂, 5 mM EGTA, 0.025% Tween-20, pH 7.4 with membranes, tracer and a dilution series of the amylin analogue. After incubation for 2 hours at room temperature the plates are centrifuged and counted on a Topcounter. The EC50 is calculated as a measure of receptor affinity.

In one aspect a protracted insulin peptide as used herein means an insulin peptide which has a time-action of more than 8 hours in standard models of diabetes. In one aspect the protracted insulin has a time-action of at least 9 hours. In a further aspect the protracted insulin has a time-action of at least 10 hours. In yet a further aspect the protracted meal-related insulin has a time-action in the range from 9 to 15 hours. In a still further aspect the protracted insulin has a time-action similar to that observed for commercial pharmaceutical compositions of insulin glargine (Lantus®).

In one aspect a protracted insulin peptide according to the invention is an insulin analogue. In a further aspect a protracted insulin peptide is an insulin derivative. In a yet further aspect a protracted insulin peptide is an insulin molecule which is acylated in one or more positions, such as in the B29 position of human insulin or desB30 human insulin.

In one aspect a protracted insulin peptide according to the invention is selected from the group consisting of: GlyA21 ArgB31 ArgB32 human insulin, NεB29-tetradecanoyl GlnB3 des(B30) human insulin), NεB29-tridecanoyl human insulin, NεB29-tetradecanoyl human insulin, NεB29-decanoyl human insulin, DesB30 ArgB31 ArgB32 human insulin and NεB29-dodecanoyl human insulin.

In one aspect a protracted insulin peptide according to the invention is GlyA21 ArgB31 ArgB32 human insulin.

GlyA21 ArgB31 ArgB32 human insulin (insulin glargine) is a protracted insulin analogue currently marketed as an acidic formulation under the trade name Lantus®. The profile of action of Lantus® is protracted due to precipitation upon injection.

In one aspect of the invention, the concentration of said insulin peptide in said pharmaceutical composition is in the range from about 1.0 mg/mL to about 5.5 mg/mL. In a further aspect the concentration of the insulin peptide is from about 1.8 mg/ml to about 5.5 mg/ml. In a yet further aspect the concentration of the insulin peptide is from about 2.5 mg/ml to about 4.5mg/ml. In a yet further aspect the concentration of the insulin peptide is from about 3 mg/ml to about 4 mg/ml. In a still further aspect the concentration of the insulin peptide is from about 3.25 mg/ml to about 3.75 mg/ml.

In a further aspect of the invention, the pH of said pharmaceutical composition or a reconstituted solution of said pharmaceutical composition is from about pH 3.0 to about pH 5.5. In a yet further aspect the pH is from about pH 3.5 to about pH 5.0. In a yet further aspect the pH is from about pH 3.5 to about pH 4.5. In a still further aspect of the invention, the pH of said pharmaceutical composition or a reconstituted solution of said pharmaceutical composition is about pH 4.

In one aspect of the invention, the pharmaceutical composition is a solution. In another aspect of the invention, the pharmaceutical composition is a solid. In another aspect of the invention, the pharmaceutical composition is to be reconstituted with an aqueous solution, such as a buffer or water for injection. In another aspect of the invention, the pharmaceutical composition is suitable for administration by injection or infusion. In a further aspect of the invention, the pharmaceutical composition is suitable for administration for subcutaneous administration. In another aspect of the invention, the pharmaceutical composition is suitable for intramuscular administration. In another aspect of the invention, the pharmaceutical composition is suitable for intravenous administration.

In one aspect of the invention, said protracted insulin peptide is GlyA21 ArgB31 ArgB32 human insulin and said amylin peptide is Pro25 Pro28 Pro29-h-amylin-h-amylin. In a further aspect of the invention, the concentration of said Pro25 Pro28 Pro29-h-amylin-h-amylin is in the range from about 0.2 mg/mL to about 1.2 mg/mL and the concentration of GlyA21 ArgB31 ArgB32 human insulin is in the range from about 1.0 mg/ml to about 5.5 mg/mL. In yet a further aspect of the invention, the pH of a composition according to the invention comprising Pro25 Pro28 Pro29-h-amylin- h-amylin and GlyA21 ArgB31 ArgB32 human insulin is in the range from about pH 3.5 to about pH 5.5.

In another aspect of the invention, said insulin peptide is GlyA21 ArgB31 ArgB32 human insulin and said amylin peptide is human amylin methylated in position 24 and 26. In a further aspect of the invention, the concentration of human amylin methylated in position 24 and 26 is in the range from about 0.2 mg/mL to about 1.2 mg/mL and the concentration of GlyA21 ArgB31 ArgB32 human insulin is in the range from about 1.0 mg/mL to about 5.5 mg/mL. In yet a further aspect of the invention, the pH of a composition according to the invention comprising human amylin methylated in position 24 and 26 and GlyA21 ArgB31 ArgB32 human insulin is in the range from about pH 3.5 to about pH 5.5.

In another aspect of the invention, said protracted insulin peptide is GlyA21 ArgB31 ArgB32 human insulin and said amylin peptide is human amylin. In a further aspect of the invention, the concentration of said human amylin is in the range from about 0.2 mg/mL to about 1.2 mg/mL and the concentration of GlyA21 ArgB31 ArgB32 human insulin is in the range from about 1.0 mg/ml to about 5.5 mg/mL. In yet a further aspect of the invention, the pH of a composition according to the invention comprising human amylin and GlyA21 ArgB31 ArgB32 human insulin is in the range from about pH 3.5 to about pH 5.5.

### Pharmaceutical Formulations

In one aspect, the present invention relates to a pharmaceutical composition which further comprises zinc. In one aspect of the invention the ratio of zinc to insulin peptide is at least 1 zinc ion per 3 insulin molecules, in another aspect the ratio is at least 1 zinc ion per 1 insulin molecule, in another aspect the ratio is at least 2 zinc ions per 3 insulin molecules, and in yet another aspect the ratio is at least 5 zinc ions per 6 insulin molecules.

The pharmaceutical compositions of the invention are chemically stable and soluble at the desired pH. By “soluble at a given pH” is meant that the insulin peptide and/or the amylin peptide contained in the composition of the invention is fully dissolved at the pH of the composition where methods for determining whether the insulin peptide and/or the amylin peptide contained in the composition of the invention are dissolved are known in the art.

In one aspect, the pharmaceutical composition may be subjected to centrifugation for 20 minutes at 30,000 g and then insulin peptide and/or the amylin peptide concentration in the supernatant may be determined by RP-HPLC. If this concentration is equal within experimental error to the insulin peptide and/or the amylin peptide concentration originally used to make the composition, then the insulin peptide and/or the amylin peptide is fully soluble in the composition of the invention.

In another aspect, the solubility of the insulin and/or the amylin peptide(s) in a composition of the invention can simply be determined by examining by eye the container in which the composition is contained. The insulin and/or the amylin peptide(s) is soluble if the solution is clear to the eye and no particulate matter is either suspended or precipitated on the sides/bottom of the container.

A basic technical problem when formulating therapeutic peptides is to obtain an acceptable shelf-life and in-use time. This requires sufficient both physical and chemical stability. Low physical stability of an amylin peptide such as wild type Amylin, may e.g. lead to amyloid fibril formation, which is observed as well-ordered, thread-like macromolecular structures in the sample eventually resulting in gel formation. The analogue pramlintide solves this problem to some extend, but still needs to be formulated as an acidic product,

Lantus is likewise formulated as an acidic, clear solution, but precipitates upon subcutaneous injection.

In one aspect a pharmaceutical composition according to the invention is physically and chemically stable. In a further aspect the pharmaceutical composition is stable at pH 3.5 to pH 5.5. In a yet further aspect the pharmaceutical composition is at least as physically and chemically stable as the least stable component of the amylin peptide and the protracted insulin, i.e. the pharmaceutical composition does not have an unacceptable lower chemical and/or physical stability than the optimal formulations of the single components alone. In one aspect of the invention a pharmaceutical formulation is obtained which is essentially free of amyloid fibrils. The amount of fibrils in a solution or suspension may be determined by a person skilled in the art by e.g. visual inspection or in a ThT fibrillation assay.

In one aspect of the invention, the pharmaceutical composition according to the invention is a “physical stable” pharmaceutically composition. The term “physical stable” as used in this context means that the amylin peptide and the protracted insulin peptide does not destabilize each other in the combined composition i.e. the pharmaceutical composition is as physical stable as the least stable of the amylin peptide and the protracted insulin peptide alone. Physical stability may be determined as described in the ThT fibrillation assay under “Methods”.

Of course, it is to be understood by the skilled artisan that the solubility of the insulin and/or the amylin peptide(s) in a composition of the invention may be affected not only by the composition and its pH but also by the temperature and time at which the composition is stored prior to measurement of solubility.

In one aspect of the invention, said pharmaceutical composition comprises a preservative.

In a further aspect of the invention, said pharmaceutical composition comprises a buffer.

In a further aspect of the invention, said pharmaceutical composition comprises an isotonicity agent.

In a further aspect of the invention, said pharmaceutical composition comprises a stabiliser.

In a further aspect of the invention, said pharmaceutical composition comprises a surfactant. As examples of surfactants mention can be made of anionic surfactants, cationic surfactants, nonionic surfactants, and zwitterionic surfactants.

In one aspect of the invention, the surfactant is an alkyl-polyglucoside.

In one aspect of the invention, said surfactant is a poloxamer.

In one aspect of the invention, said surfactant is a polysorbate (Tween).

In another aspect, the invention relates to a method for treatment of hyperglycemia by parenteral administration of an effective amount of a pharmaceutical composition, which comprises an amylin peptide, and a protracted insulin peptide.

In another aspect, the present invention relates to a method for treatment of binge eating or bulimia comprising parenteral administration of an effective amount of a pharmaceutical composition, which comprises an amylin peptide, and a protracted insulin peptide.

In another aspect, the present invention relates to a method for treatment or prevention of type 2 diabetes, impaired glucose tolerance, type 1 diabetes, obesity, hypertension, syndrome X, dyslipidemia, cognitive disorders, atheroschlerosis, myocardial infarction, coronary heart disease and other cardiovascular disorders, stroke, inflammatory bowel syndrome, dyspepsia and gastric ulcers comprising parenteral administration of an effective amount of a pharmaceutical composition, which comprises an amylin peptide, and a protracted insulin peptide.

In another aspect, the present invention relates to a method for delaying or preventing disease progression in type 2 diabetes comprising parenteral administration of an effective amount of a pharmaceutical composition, which comprises an amylin peptide, and a protracted insulin peptide.

In another aspect, the present invention relates to a method for decreasing food intake, decreasing β-cell apoptosis, increasing β-cell function and β-cell mass, and/or for restoring glucose sensitivity to β-cells comprising parenteral administration of an effective amount of a pharmaceutical composition, which comprises an amylin peptide, and a protracted insulin peptide.

In a further aspect the present invention relates to a method of treating any of the above conditions which further comprises administering to a person in need thereof a pharmaceutically relevant amount of GLP-1 or a GLP-1 derivative. In another aspect the GLP-1 derivative to be employed in combination with a composition of the present invention refers to GLP-1(1-37), exendin-4(1-39), insulinotropic fragments thereof, insulinotropic analogues thereof and insulinotropic derivatives thereof. Insulinotropic fragments of GLP-1(1-37) are insulinotropic peptides for which the entire sequence can be found in the sequence of GLP-1(1-37) and where at least one terminal amino acid has been deleted. Examples of insulinotropic fragments of GLP-1(1-37) are GLP-1(7-37) wherein the amino acid residues in positions 1-6 of GLP-1(1-37) have been deleted, and GLP-1(7-36) where the amino acid residues in position 1-6 and 37 of GLP-1(1-37) have been deleted. Examples of insulinotropic fragments of exendin-4(1-39) are exendin-4(1-38) and exendin-4(1-31). The insulinotropic property of a compound may be determined by in vivo or in vitro assays well known in the art. For instance, the compound may be administered to an animal and monitoring the insulin concentration overtime. Insulinotropic analogues of GLP-1(1-37) and exendin-4(1-39) refer to the respective molecules wherein one or more of the amino acids residues have been exchanged with other amino acid residues and/or from which one or more amino acid residues have been deleted and/or from which one or more amino acid residues have been added with the proviso that said analogue either is insulinotropic or is a prodrug of an insulinotropic compound . Examples of insulinotropic analogues of GLP-1(1-37) are e.g. Met⁸-GLP-1(7-37) wherein the alanine in position 8 has been replaced by methionine and the amino acid residues in position 1 to 6 have been deleted, and Arg34-GLP-1(7-37), wherein the valine in position 34 has been replaced with arginine and the amino acid residues in position 1 to 6 have been deleted. An example of an insulinotropic analogue of exendin-4(1-39) is Ser²Asp³-exendin-4(1-39) wherein the amino acid residues in position 2 and 3 have been replaced with serine and aspartic acid, respectively (this particular analogue also being known in the art as exendin-3). Insulinotropic derivatives of GLP-1(1-37), exendin-4(1-39) and analogues thereof are what the person skilled in the art considers to be derivatives of these peptides, i.e. having at least one substituent which is not present in the parent peptide molecule with the proviso that said derivative either is insulinotropic or is a prodrug of an insulinotropic compound. Examples of substituents are amides, carbohydrates, alkyl groups and lipophilic substituents. Examples of insulinotropic derivatives of GLP-1(1-37), exendin-4(1-39) and analogues thereof are GLP-1(7-36)-amide, Arg³⁴, Lys²⁸(N^{ε}-(γ-Glu(N^{α}-hexadecanoyl)))-GLP-1(7-37) and Tyr³¹-exendin-4(1-31)-amide. Further examples of GLP-1(1-37), exendin-4(1-39), insulinotropic fragments thereof, insulinotropic analogues thereof and insulinotropic derivatives thereof are described in WO 98/08871 (Novo Nordisk A/S), WO 99/43706 (Novo Nordisk A/S, U.S. Pat. No. 5,424,286 (Eng), WO 00/09666 (The Government of the USA), WO 2006/097537 (Novo Nordisk A/S) and European Patent application No. 08101008.4 (Novo Nordisk NS).

When the pharmaceutical compositions according to the present invention are administered by injection, e.g. via a pen or a syringe, it is typically administered 3 times per day, preferably before meals. It is preferred that each administration comprises less than 1 ml, optionally less than 750 μl since larger injection volumes are unpleasant for the patient.

In one aspect of the invention, the method of treatment comprises administration of an effective amount of the pharmaceutical composition which is from about 30 μL/day to about 800 μL/day, such as from about 60 μL/day to about 360 μL/day. In another aspect of the invention the method comprises a pharmaceutical composition for administration by subcutaneous injection.

In another aspect the present invention, relates to a pharmaceutical composition comprising an amylin peptide and a protracted insulin peptide for use as a medicament for parenteral administration. In one aspect of the invention, the pharmaceutical composition is for use as a medicament for administration by subcutaneous injection. In another aspect, the present invention relates to a pharmaceutical composition comprising an amylin peptide and a protracted insulin peptide for use as a medicament in the treatment of hyperglycemia by parenteral administration.

In another aspect, the present invention relates to a pharmaceutical composition according to the invention for use as a medicament in the treatment of hyperglycemia. In another aspect, the present invention relates to a pharmaceutical composition comprising an amylin peptide and a protracted insulin peptide for use as a medicament for the treatment of binge eating or bulimia.

### Further Aspects of the Invention

1. A soluble pharmaceutical composition for parenteral administration, which comprises an amylin peptide, and a protracted insulin peptide.

2. The pharmaceutical composition as defined in aspect 1, wherein the pH of said pharmaceutical composition or a reconstituted solution of said pharmaceutical composition is from about pH 3.5 to about pH 5.5

3. The pharmaceutical composition as defined in any of the aspects 1 or 2, wherein the composition is a solution.

4. The pharmaceutical composition as defined in any of the aspects 1-3, wherein the composition is a solid.

5. The pharmaceutical composition as defined in aspect 4, which is to be reconstituted with an aqueous solution, such as a buffer or water for injection.

6. The pharmaceutical composition as defined in any of the aspects 1-5, which is suitable for administration by injection or infusion.

7. The pharmaceutical composition as defined in any of the aspects 1-6, wherein said protracted insulin peptide has a time-action of more than 8 hours.

8. The pharmaceutical composition as defined in any of the aspects 1-7, wherein said protracted insulin peptide is GlyA21 ArgB31 ArgB32 human insulin.

9. The pharmaceutical composition as defined in any of the aspects 1-8, wherein the concentration of said protracted insulin peptide is in the range from about 1.0 mg/mL to about 5.5 mg/mL, or from about 1.8 mg/mL to about 5.5 mg/mL, or from about 2.5 mg/mL to about 4.5 mg/mL, or from about 3 mg/mL to about 4 mg/mL, or from about 3.25 to about 3.75.

10. The pharmaceutical composition as defined in any of the aspects 1-9, wherein the concentration of said protracted insulin peptide is in the range from about 1.6 mg/mL to 2.0 mg/mL

11. The pharmaceutical composition as defined in any of the aspects 1-10, comprising two different insulin peptides.

12. The pharmaceutical composition as defined in any of the aspects 1-11, wherein said amylin peptide is human amylin, an amylin analogue or an amylin agonist.

13. The pharmaceutical composition as defined in any of the aspects 1-12, wherein said amylin peptide is human amylin.

14. The pharmaceutical composition as defined in any of the aspects 1-12, wherein said amylin peptide is Pro25 Pro28 Pro29-h-amylin.

15. The pharmaceutical composition as defined in any of the aspects 1-12, wherein said amylin peptide is human amylin methylated in position 24 and 26.

16. The pharmaceutical composition as defined in any of the aspects 1-12, wherein said protracted insulin peptide is GlyA21 ArgB31 ArgB32 human insulin and said amylin peptide is Pro25 Pro28 Pro29-h-amylin.

17. The pharmaceutical composition as defined in any of the aspects 1-16, wherein the concentration of said amylin peptide is in the range from about 0.05 mg/mL to about 10 mg/mL or from about 0.1 mg/mL to about 4 mg/mL, or from about 0.2 mg/mL to about 1.2 mg/mL, or from about 0.5 mg/mL to about 0.7 mg/mL.

18. The pharmaceutical composition as defined in any of the aspects 1-17, wherein the concentration of said amylin peptide is in the range from about 0.2 mg/mL to about 0.7 mg/mL or from about 0.2 mg/mL to about 0.4 mg/mL.

19. The pharmaceutical composition as defined in aspect 16, wherein the concentration of Pro25 Pro28 Pro29-h-amylin is in the range from about 0.2 mg/mL to about 1.2 mg/mL and the concentration of GlyA21 ArgB31 ArgB32 human insulin is in the range from about 1.0 mg/mL to about 5.5 mg/mL.

20. The pharmaceutical composition as defined in any of the aspects 1-19, comprising zinc.

21. The pharmaceutical composition as defined in aspect 20, wherein the ratio of zinc to insulin peptide is at least 1 zinc ion per 3 insulin molecules, or at least 1 zinc ion per 1 insulin, or at least 2 zinc ions per 3 insulin, or at least 5 zinc ions per 6 insulin molecules.

22. The pharmaceutical composition as defined in any of the aspects 1-21, wherein said pharmaceutical composition comprises a preservative.

23. The pharmaceutical composition as defined in any of the aspects 1-22, wherein said pharmaceutical composition comprises a buffer.

24. The pharmaceutical composition as defined in any of the aspects 1-23, wherein said pharmaceutical composition comprises an isotonicity agent.

25. The pharmaceutical composition as defined in any of the aspects 1-24, which further comprises a stabiliser.

26. The pharmaceutical composition as defined in any of the aspects 1-25, which further comprises a surfactant.

27. The pharmaceutical composition as defined in aspect 16, wherein the pH of said pharmaceutical composition or a reconstituted solution of said pharmaceutical composition is from about pH 3.5 to about pH 5.5.

28. A method for treatment of hyperglycemia comprising parenteral administration of an effective amount of the pharmaceutical composition as defined in any of the aspects 1-27.

29. The method as defined in aspect 28, wherein said effective amount of the pharmaceutical composition is from about 30 μL/day to about 600 μL/day, such as from about 60 μL/day to about 360 μL/day, or from about 250 μl/day to about 750 μl/day.

30. A pharmaceutical composition comprising a protracted insulin peptide and an amylin peptide as defined in any of the aspects 1-27 for use as a medicament.

31. A composition comprising a protracted insulin peptide and an amylin peptide as defined in any of the aspects 1-30 for use as a medicament in the treatment of hyperglycemia.

32. A composition comprising a protracted insulin peptide and an amylin peptide as defined in any of the aspects 1-30 for the use as a pharmaceutical composition in the treatment of binge eating or bulimia.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### Examples

### General Introduction to ThT Fibrillation Assays for the Assessment of Physical Stability of Protein Formulations

Low physical stability of a peptide may lead to amyloid fibril formation, which is observed as well-ordered, thread-like macromolecular structures in the sample eventually resulting in gel formation. This has traditionally been measured by visual inspection of the sample. However, that kind of measurement is very subjective and depending on the observer. Therefore, the application of a small molecule indicator probe is much more advantageous. Thioflavin T (ThT) is such a probe and has a distinct fluorescence signature when binding to fibrils [Naiki et al. (1989) Anal. Biochem. 177, 244-249; LeVine (1999) Methods. Enzymol. 309, 274-284].

The time course for fibril formation can be described by a sigmoidal curve with the following expression [Nielsen et al. (2001) Biochemistry 40, 6036-6046]:

$$F = {f}_{i} + {m}_{i} t + \frac{{f}_{f} + {m}_{f} t}{1 + {e}^{- [ ( t - {t}_{0} ) / τ ]}} \qquad Eq . ( 1 )$$

Here, F is the ThT fluorescence at the time t. The constant t₀ is the time needed to reach 50% of maximum fluorescence. The two important parameters describing fibril formation are the lag-time calculated by t₀−2τ and the apparent rate constant kₐₚₚ=1/τ.

Formation of a partially folded intermediate of the peptide is suggested as a general initiating mechanism for fibrillation. Few of those intermediates nucleate to form a template onto which further intermediates may assembly and the fibrillation proceeds. The lag-time corresponds to the interval in which the critical mass of nucleus is built up and the apparent rate constant is the rate with which the fibril itself is formed.

### Sample Preparation

Samples were prepared freshly before each assay. Each sample composition is described in each example. The pH of the sample was adjusted to the desired value using appropriate amounts of concentrated NaOH and HClO₄ or HCl. Thioflavin T was added to the samples from a stock solution in H₂O to a final concentration of 1 μM.

Sample aliquots of 200 μl were placed in a 96 well microtiter plate (Packard OptiPlate™-96, white polystyrene). Usually, four or eight replica of each sample (corresponding to one test condition) were placed in one column of wells. The plate was sealed with Scotch Pad (Qiagen).

### Incubation and Fluorescence Measurement

Incubation at given temperature, shaking and measurement of the ThT fluorescence emission were done in a Fluoroskan Ascent FL fluorescence platereader or Varioskan platereader (Thermo Labsystems). The temperature was adjusted to 37° C. The orbital shaking was adjusted to 960 rpm with an amplitude of 1 mm in all the presented data. Fluorescence measurement was done using excitation through a 444 nm filter and measurement of emission through a 485 nm fitter.

Each run was initiated by incubating the plate at the assay temperature for 10 min. The plate was measured every 20 minutes for a desired period of time. Between each measurement, the plate was shaken and heated as described.

### Data Handling

The measurement points were saved in Microsoft Excel format for further processing and curve drawing and fitting was performed using GraphPad Prism. The background emission from ThT in the absence of fibrils was negligible. The data points are typically a mean of four or eight samples and shown with standard deviation error bars. Only data obtained in the same experiment (i.e. samples on the same plate) are presented in the same graph ensuring a relative measure of fibrillation between experiments.

The data set may be fitted to Eq. (1). However, since full sigmodial curves in this case are not always achieved during the measurement time, the degree of fibrillation is expressed as ThT fluorescence tabulated as the mean of the samples and shown with the standard deviation at various time points.

### Luciferase Assay

1. Amylin Assay Outline

It has previously been published (Poyner DR et al 2002, Pharmacological Reviews 54(2) 233-246) that activation of Amylin receptors (coexpression of Calcitonin receptor and receptor activity modifying peptides RAMPs) by Amylin leads to an increase in the intracellular concentration of cAMP. Consequently, transcription is activated at promotors containing multiple copies of the cAMP response element (CRE). It is thus possible to measure Amylin activity by use of a CRE luciferase reporter gene introduced into BHK cells also expressing an Amylin receptor.

2. Construction of an Amylin 3(a)/CRE-luc Cell Line

A BHK570 cell line stably transfected with the human calcitonin receptor (CTa) and a CRE-responsive luciferase reportergene. The cell line was further transfected with RAMP-3, using standard methods. This turns the Calcitonin receptor into an Amylin 3(a) receptor. Methotrexate, Neomycin, and Hygromycin are selection markers for luciferase, the Calcitonin receptor, and RAMP-3, respectively.

3. Amylin Luciferase Assay

To perform activity assays, BHK Amylin 3(a)//CRE-luc cells were seeded in white 96 well culture plates at a density of about 20.000 cells/well. The cells were in 100 μl growth medium (DMEM with 10% FBS, 1% Pen/Strep, 1 mM Na-pyruvate, 250 nM Methotrexate, 500 μg/ml Neomycin, and 400 μg/ml Hygromycin). After incubation overnight at 37° C. and 5% CO₂, the growth medium was replaced by 50 μl/well assay medium (DMEM (without phenol red), Glumamax™, 10% FBS, and 10 mM Hepes, pH 7,4). Further, 50 μl/well of standard or sample in assay buffer were added. After 4 hours incubation at 37° C. and 5% CO₂, the assay medium with standard or sample were removed and replaced by 100 μl/well PBS. Further, 100 μl/well LucLite™ was added. The plates were sealed and incubated at room temperature for 30 minutes. Finally, luminescence was measured on a TopCounter (Packard) in SPC (single photon counting) mode.

### General Introduction to Protein Solubility

The solubility of peptides and proteins depends on the pH of the solution. Often a protein or peptide precipitates at and/or close to its isoelectric point (pl), at which its netto charge is zero. At pH lower than the pl proteins and peptides are typically positively charged, at pH higher than the pl they are negatively charged.

A prerequisite for mixing insulin and amylin is that both peptides remain soluble at a given pH, which is suitable for both formulating the drug product and for administrating the drug product to the patient e.g. by subcutaneous injection.

Solubility versus pH curves were measured in the following way. A formulation was prepared and aliquotes were adjusted to pH values in the desired range by adding HClO₄ or HCl and NaOH. These samples were left equilibrating at room temperature for 2-3 days. Then the samples were centrifuged. A small aliquot of each sample was withdrawn for reverse HPLC analysis for determination of the concentration of the proteins in solution. The pH of each sample was measured after the centrifugation, and the concentration of each protein was depicted versus the measured pH.

### Example 1

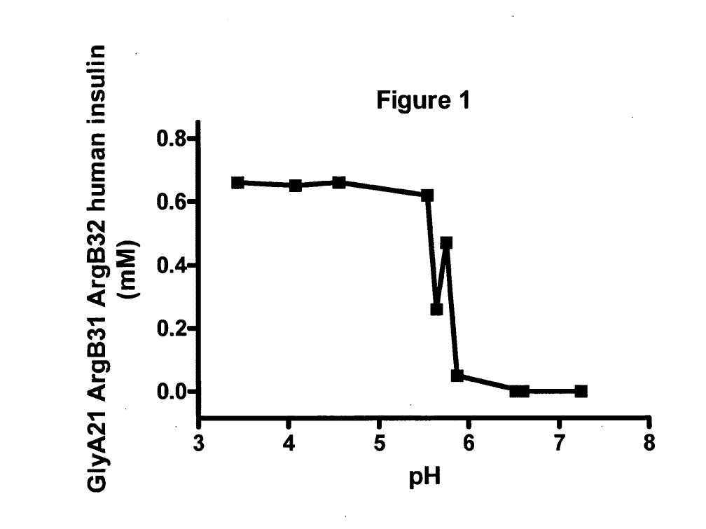

FIG. 1 shows the solubility of a formulation of GlyA21 ArgB31 ArgB32 human insulin versus pH. The formulation consisted of 0.6 mM GlyA21 ArgB31 ArgB32 human insulin, 0.46 mM Zn(Ac)₂, 30 mM phenol. The GlyA21 ArgB31 ArgB32 human insulin analogue completely precipitated below physiological pH (pH 7.4). Complete precipitation on the injection site is the protraction principle for this insulin analogue and is due to addition of the two arginines in positions B31 and B32. Furthermore, the substitution of residue asparagine A21 to glycine confers chemical stability when formulating the analogue at acidic pH (e.g. pH 4.0) in order to obtain a fully soluble drug product.

### Example 2

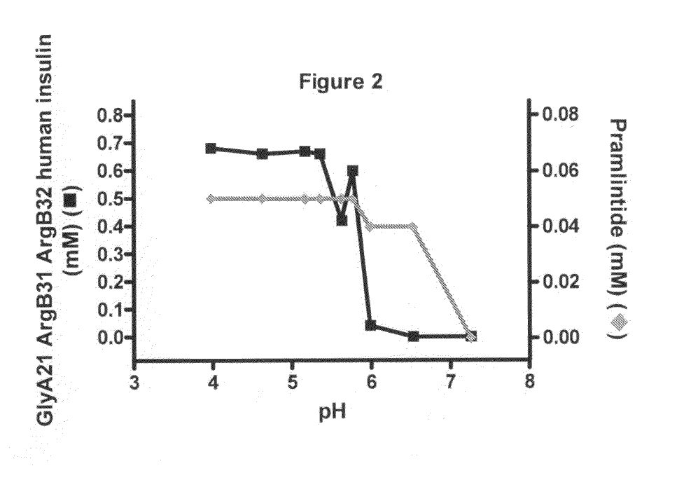

FIG. 2 shows the solubility of a mix formulation of GlyA21 ArgB31 ArgB32 human insulin and the amylin analogue pramlintide versus pH. The mix formulation consisted of 0.6 mM GlyA21 ArgB31 ArgB32 human insulin, 0.46 mM Zn(Ac)₂, 30 mM phenol, 50 μM pramlintide. The concentration of GlyA21 ArgB31 ArgB32 human insulin in solution versus pH was plotted with black lines and squares using the left y-axis; the concentration of pramlintide in solution versus pH was plotted with light grey lines and diamonds using the right y-axis. The precipitation of GlyA21 ArgB31 ArgB32 human insulin was not changed compared to the formulation of the insulin analogue alone as in Example 1. Furthermore, pramlintide also fully precipitated below physiological pH (pH 7.4). Usually pramlintide is soluble at pH 7.4. The experiment indicated that pramlintide coprecipitated together with GlyA21 ArgB31 ArgB32 human insulin without changing the precipitation of the insulin analogue. It is thus indicated that the protraction and pharmacokinetic properties of GlyA21 ArgB31 ArgB32 human insulin are unaltered. Furthermore, the precipitation of pramlintide could possibly prolong its activity and result in an advantageous longer duration.

### Example 3

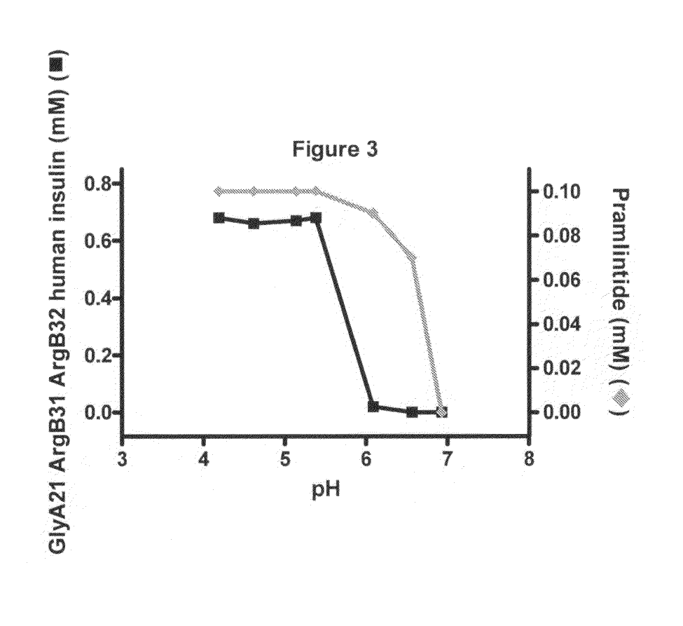

FIG. 3 shows the solubility of a mix formulation of GlyA21 ArgB31 ArgB32 human insulin and pramlintide versus pH. The mix formulation consisted of 0.6 mM GlyA21 ArgB31 ArgB32 human insulin, 0.3 mM Zn(Ac)₂, 30 mM phenol, 100 μM pramlintide. The concentration of GlyA21 ArgB31 ArgB32 human insulin in solution versus pH was plotted with black lines and squares using the left y-axis; the concentration of pramlintide in solution versus pH was plotted with light grey lines and diamonds using the right y-axis. Both GlyA21 ArgB31 ArgB32 human insulin and pramlintide were completely precipitated at pH 7 indicating unchanged protraction of GlyA21 ArgB31 ArgB32 human insulin. Furthermore, the coprecipitation of pramlintide with the insulin analogue may prolong its activity.

### Example 4

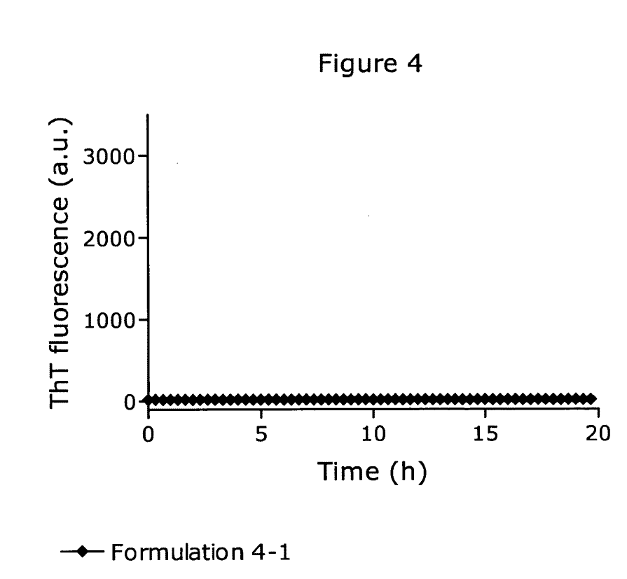

The physical stability of a pramlintide formulation similar to the commercially available Symlin® formulation was assessed using a ThT fibrillation assay as shown in FIG. 4. Formulation 4-1 consisted of 150 μM pramlintide, 0.6 g/l sodium-acetate, 1.5 g/l acetic acid (a total acetate concentration of 1.77 g/l), 236 mM mannitol, 20 mM m-cresol, adjusted to pH 4.0. Under the applied conditions the formulation was inert towards fibrillation as no ThT fluorescence signal emerged. The mean ThT fluorescence is tabulated below at certain time points

| | 5 h | SD | 15 h | SD |
|---|---|---|---|---|
| Formulation 4-1 | 18 | 0 | 18 | 0 |

### Example 5

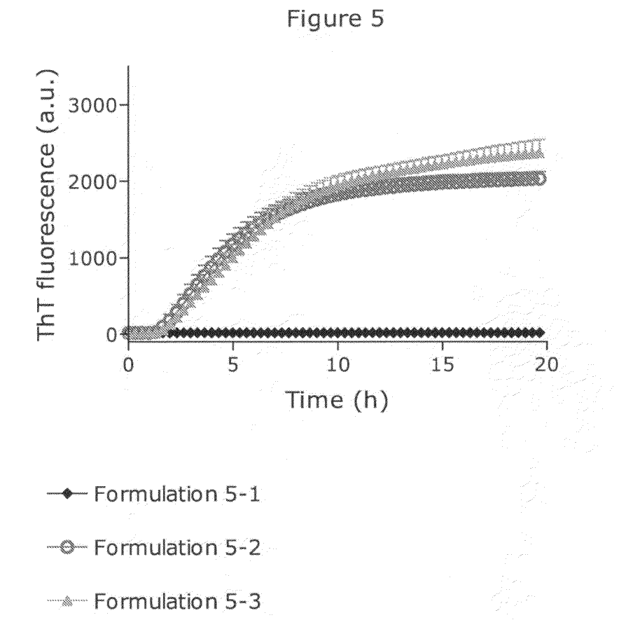

The physical stability of a pramlintide formulation, an GlyA21 ArgB31 ArgB32 human insulin formulation and a mix formulation containing both pramlintide and GlyA21 ArgB31 ArgB32 human insulin was compared using the ThT fibrillation assay. Formulation 5-1 consisted of 100 μM pramlintide, 185 mM glycerol, 25 mM phenol, adjusted to pH 4.0. Formulation 5-2 consisted of 0.6 mM GlyA21 ArgB31 ArgB32 human insulin, 185 mM glycerol, 25 mM phenol, adjusted to pH 4.0. Formulation 5-3 consisted of 0.6mM GlyA21 ArgB31 ArgB32 human insulin, 100 μM pramlintide, 185 mM glycerol, 25 mM phenol, adjusted to pH 4.0. The results are shown in FIG. 5. The pramlintide formulation (5-1) was inert towards fibrillation. The GlyA21 ArgB31 ArgB32 human insulin formulation (5-2) initiated fibrillation with a lag time of approximately 1.3 hours. The mix formulation containing both GlyA21 ArgB31 ArgB32 human insulin and pramlintide (5-3) also initiated fibrillation with a lag time similar to that of GlyA21 ArgB31 ArgB32 human insulin alone. Hence, there was no mutual destabilisation since the mixture was equally physically stable as the least stable component (GlyA21 ArgB31 ArgB32 human insulin) alone. The mean ThT fluorescence at various time points is shown below for the three formulations.

| | 1 h | SD | 5 h | SD | 15 h | SD |
|---|---|---|---|---|---|---|
| Formulation 5-1 | 18 | 1 | 17 | 1 | 17 | 1 |
| Formulation 5-2 | 18 | 1 | 1167 | 144 | 1979 | 97 |
| Formulation 5-3 | 18 | 1 | 1005 | 167 | 2230 | 106 |

### Example 6

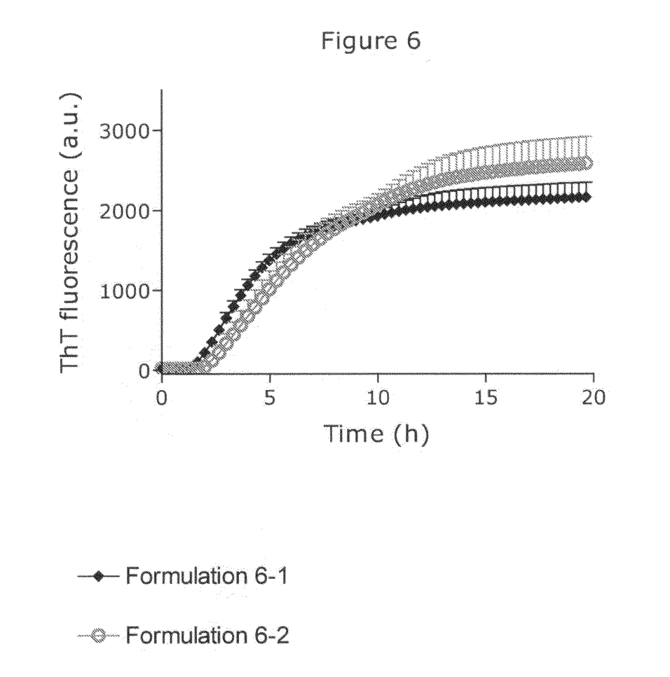

The physical stability of two GlyA21 ArgB31 ArgB32 human insulin formulations without and with pramlintide was compared in a ThT fibrillation assay. This comparison is shown in FIG. 6. Formulation 6-1 consisted of 0.6mM GlyA21 ArgB31 ArgB32 human insulin, 0.46 mM Zn(Ac)₂, 185 mM glycerol, 25 mM phenol, adjusted to pH 4.0. Formulation 6-2 consisted of 0.6mM GlyA21 ArgB31 ArgB32 human insulin, 100 μM pramlintide, 0.46 mM Zn(Ac)₂, 185 mM glycerol, 25 mM phenol, adjusted to pH 4.0. There was no significant difference in the lag times before fibrillation of the two formulations. Hence, there was no mutual destabilisation since the mix formulation was just as physically stable as the formulation of GlyA21 ArgB31 ArgB32 human insulin alone. The mean ThT fluorescence at various time points is shown below for the two formulations.

| | 1 h | SD | 5 h | SD | 15 h | SD |
|---|---|---|---|---|---|---|
| Formulation 6-1 | 18 | 1 | 1372 | 78 | 2102 | 179 |
| Formulation 6-2 | 18 | 1 | 1009 | 231 | 2471 | 320 |

1. A soluble pharmaceutical composition for parenteral administration, which comprises an amylin peptide, and a protracted insulin peptide.

2. The pharmaceutical composition as defined in claim 1, wherein the pH of said pharmaceutical composition or a reconstituted solution of said pharmaceutical composition is from about pH 3.5 to about pH 5.5

3. The pharmaceutical composition according to claim 2, wherein said protracted insulin peptide has a time-action of more than 8 hours.

4. The pharmaceutical composition according to claim 3, wherein said protracted insulin peptide is GlyA21 ArgB31 ArgB32 human insulin.

5. The pharmaceutical composition according to claim 4, wherein the concentration of said protracted insulin peptide is in the range from about 1.0 mg/mL to about 5.5 mg/mL.

6. The pharmaceutical composition according to claim 1, wherein said amylin peptide is human amylin, an amylin analogue or an amylin agonist.

7. The pharmaceutical composition according to claim 6, wherein the concentration of said amylin peptide is in the range from about 0.05 mg/mL to about 10 mg/mL.

8. The pharmaceutical composition according to claim 1, wherein said protracted insulin peptide is GlyA21 ArgB31 ArgB32 human insulin and said amylin peptide is Pro25 Pro28 Pro29-h-amylin.

9. A method for treatment of hyperglycemia in a subject in need of such treatment, the method comprising parenteral administration to the subject of a a therapeutically effective amount of the pharmaceutical composition according to claim 1.

10-12. (canceled)

13. A method for treatment of binge eating or bulimia in a subject in need of such treatment, the method comprising parenteral administration to the subject of a therapeutically effective amount of the pharmaceutical composition according to claim 1.
